(19) United States
(12) Patent Application Publication
(10) Pub. No.: US 2025/0027876 A1
(43) Pub. Date: Jan. 23, 2025

(54) DETECTING AND DIFFERENTIATING NEUROTRANSMITTERS USING ULTRAVIOLET PLASMONIC-ENGINEERED NATIVE FLUORESCENCE

(71) Applicant: UNIVERSITY OF UTAH RESEARCH FOUNDATION, Salt Lake City, UT (US)

(72) Inventors: Yunshan Wang, Salt Lake City, UT (US); Ji-Young Lee, Salt Lake City, UT (US)

(21) Appl. No.: 18/774,407

(22) Filed: Jul. 16, 2024

Related U.S. Application Data
(60) Provisional application No. 63/514,183, filed on Jul. 18, 2023.

(51) Int. Cl. G01N 21/33; G01N 21/552; G01N 33/94

(52) U.S. Cl. CPC G01N 21/33; G01N 21/553; G01N 33/9406; G01N 2333/70571

(57) ABSTRACT

Method and apparatus for detecting and differentiating neurotransmitters using ultraviolet plasmonic-engineered native fluorescence. In one example, the method includes determining a photobleaching rate constant of a neurotransmitter-containing analyte loaded onto a plasmonic-engineered biosensor and subjected to illumination by ultraviolet light. The method further includes submitting a query containing the determined rate constant to a database including calibration data representing a plurality of different neurotransmitters and a plurality of different biosensors. In at least some examples, the queried database returns a response indicating one or more of a predicted identity of the neurotransmitter together with a corresponding confidence score, an estimated amount of the neurotransmitter in the analyte, and an estimated percentage of the neurotransmitter relative to another neurotransmitter in the analyte.

### CROSS-REFERENCE TO RELATED APPLICATIONS

This application claims the benefit of U.S. Provisional Patent Application No. 63/514,183 filed on Jul. 18, 2023, and entitled “Detecting and Differentiating Neurotransmitters Using Ultraviolet Plasmonic Engineered Native Fluorescence,” the contents of which are incorporated herein by reference.

### FIELD OF THE DISCLOSURE

Various example embodiments relate to analytical chemistry and, more specifically but not exclusively, to spectroscopic methods and apparatus for detecting neurotransmitters.

### BACKGROUND

Monoamine neurotransmitters (MANTs) play an important role in the endocrine and central nervous systems. Fluctuations in the levels of MANTs may be caused by certain neurological or immunological diseases, including but not limited to the Parkinson's disease, human immunodeficiency virus infection, and schizophrenia. The physiological concentrations of MANTs in the bodily fluids are relatively low, e.g., typically being in the hundreds of picomolar (pM) to low nanomolar (nM) range. Also, different MANTs and their derivatives with similar molecular structures but different physiological functions may coexist in the bodily fluids.

### BRIEF SUMMARY OF SOME SPECIFIC EMBODIMENTS

Various examples provide methods and apparatus designed to use photobleaching of ultraviolet (UV) plasmonic-engineered native fluorescence of neurotransmitters as a sensing mechanism to achieve highly sensitive and specific detection of neurotransmitters. In one example, an analyte under test (AUT) containing a neurotransmitter is loaded onto a plasmonic-engineered biosensor and is subjected to illumination by ultraviolet light that causes both fluorescence and photochemical reactions of the neurotransmitter. A time series of the fluorescence spectra is collected with a fluorimeter and is processed with a computing device to determine a corresponding set of photobleaching parameters. A query containing some or all of the determined photobleaching parameters along with pertinent metadata is then submitted to a database that has photobleaching calibration data representing multiple neurotransmitters and multiple biosensor variants. In at least some examples, the queried database returns a response indicating one or more of: (i) a predicted identity of the neurotransmitter together with the corresponding confidence score, (ii) an estimated amount of the neurotransmitter in the AUT, and (iii) an estimated percentage amount of the neurotransmitter relative to another neurotransmitter in the AUT. Various embodiments are beneficially capable of detecting, differentiating, and/or quantifying neurotransmitters in AUTs without the need for an aptamer, antibody, chemical reaction, or enzymatic reaction and are expected to be of significant practical interest to various biomedical applications dealing with neurotransmitters.

In one example, an analytical apparatus comprises: a fluorimeter configured to measure a time series of fluorescence spectra of a biosensor having loaded thereon an analyte and subjected to illumination by ultraviolet light, the biosensor including an engineered layer of plasmonic material, the analyte including a neurotransmitter, different ones of the fluorescence spectra in the time series corresponding to different respective illumination times; and a computing device configured to: determine a first rate constant based on the time series, the first rate constant corresponding to a photochemical reaction of the neurotransmitter caused by the ultraviolet light; submit to a database a query containing at least the determined first rate constant, the database including photobleaching calibration data representing a plurality of different neurotransmitters and a plurality of different biosensors; and display on a display device a response to the submitted query received from the database.

In another example, an analytical method comprises: determining a first rate constant based on a time series of fluorescence spectra of a biosensor having loaded thereon an analyte and subjected to illumination by ultraviolet light, the biosensor including an engineered layer of plasmonic material, the analyte including a neurotransmitter, the first rate constant corresponding to a photochemical reaction of the neurotransmitter caused by the ultraviolet light; submitting to a database a query containing at least the determined first rate constant, the database including photobleaching calibration data representing a plurality of different neurotransmitters and a plurality of different biosensors; and receiving from the database a response to the submitted query.

### BRIEF DESCRIPTION OF THE DRAWINGS

Other aspects, features, and benefits of various disclosed embodiments will become more fully apparent, by way of example, from the following detailed description and the accompanying drawings, in which:

FIGS. 1A-1B are schematic diagrams illustrating an optical system with which various embodiments can be practiced.

FIG. 2 schematically illustrates the chemical structures of several neurotransmitter molecules that can be loaded on the biosensor of FIG. 1B according to some examples.

FIGS. 3A-3C pictorially and graphically illustrate the biosensor of FIG. 1B according to one example.

FIGS. 4A-4C graphically illustrate fluorescence spectra of several neurotransmitters obtained with the optical system of FIG. 1 according to some examples.

FIG. 5 graphically illustrates the net native fluorescence enhancement for several neurotransmitter molecules achieved with different biosensors according to some examples.

FIG. 6 graphically illustrates the kinetics of native-fluorescence decay observed for dopamine (DA) on the biosensor of FIGS. 3A-3C according to one example.

FIGS. 7A and 7B are tables illustrating sets of photobleaching parameters for several neurotransmitter molecules on several different biosensors according to some examples.

FIG. 8 graphically compares the first photobleaching rate constants, k₁, of several neurotransmitter molecules on several different biosensors according to some examples.

FIG. 9 graphically illustrates the relative-concentration dependence of the first photobleaching rate constant for a mixture of two neurotransmitters according to one example.

FIG. 10 is a flowchart illustrating a method of detecting and differentiating neurotransmitters according to some examples.

FIG. 11 is a block diagram illustrating a computing device used in the optical system of FIG. 1 according to some examples.

### DETAILED DESCRIPTION

Example analytical techniques for detecting, differentiating, and/or quantifying MANTs include liquid chromatography/mass spectroscopy (LC-MS), fast-scan cyclic voltammetry (FSCV), and nanomaterial-based biosensors. LC-MS provides relatively high sensitivity and selectivity but typically involves extensive sample preparation. In some cases, such sample preparation may disadvantageously result in an unacceptably high percentage of sample loss. FSCV can be carried out with relatively little sample preparation and can detect MANTs in vivo. However, in some cases, the FSCV techniques may not be sufficiently selective because different MANT molecules may have overlapping redox potentials. In many examples, nanomaterial-based biosensors rely on aptamers, antibodies, enzymatic reactions, or chemical reactions for selective MANT detection. However, the corresponding selection of highly specific aptamers or antibodies can be challenging and/or time-consuming in its own right, and nonspecific binding to interfering molecules may still occur even with carefully selected aptamers. Enzymatic or chemical reactions, although specific, may suffer from relatively low sensitivity.

Intrinsic-fluorescence-based sensors can be used in environmental monitoring, cell imaging, biomedical applications, and analytical chemistry. For example, protein arrays can be detected using time-resolved ultraviolet (UV) native fluorescence. In some examples, multiphoton excitation of native protein fluorescence can be used to discriminate ligands with different binding affinities. However, the relatively low quantum yield and the unstable nature of intrinsic fluorescence of biomolecules may typically constrain the usage of at least some intrinsic-fluorescence-based biosensors.

At least some of the above-indicated problems in the state of the art can beneficially be addressed using the analytical techniques for detecting and differentiating neurotransmitters with relatively high sensitivity and specificity disclosed herein. For example, we realized that the use of UV plasmonic-enhanced native fluorescence could improve the sensitivity and lower the limits of detection for at least some UV biosensors. Accordingly, some examples disclosed herein use UV plasmonic-engineered native fluorescence of neurotransmitters as a sensing mechanism to achieve highly sensitive and specific detection of at least some neurotransmitters. In some examples, an aluminum (Al) hole array achieves a net enhancement of 50× for tryptophan. In some other examples, similar enhancement factors are achieved for dopamine (DA), norepinephrine (NE), and 3,4-dihydroxyphenylacetic acid (DOPAC) on engineered Al hole arrays. We also observe that DA, NE and DOPAC have distinct photobleaching rates under UV illumination, and the pairwise differences in their photobleaching rates can be enlarged on at least some plasmonic-engineered substrates, such as an Al thin film and an Al hole array. Some embodiments therefore use the photobleaching rates of UV plasmonic-engineered native fluorescence as a mechanism for differentiating neurotransmitters without the need for an aptamer, antibody, chemical reaction, or enzymatic reaction. At least some embodiments disclosed herein are expected to be of significant practical interest in various applications involving detection, differentiation, and/or quantification of neurotransmitters.

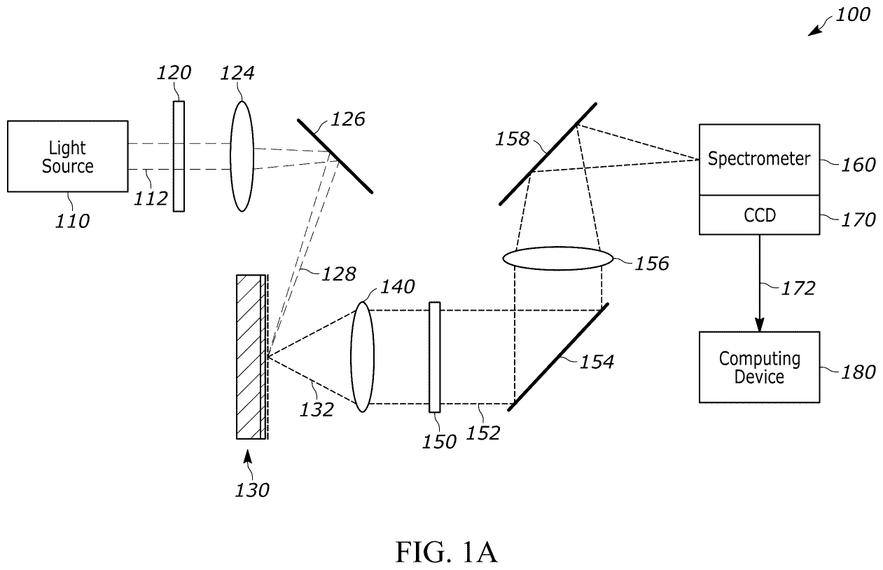

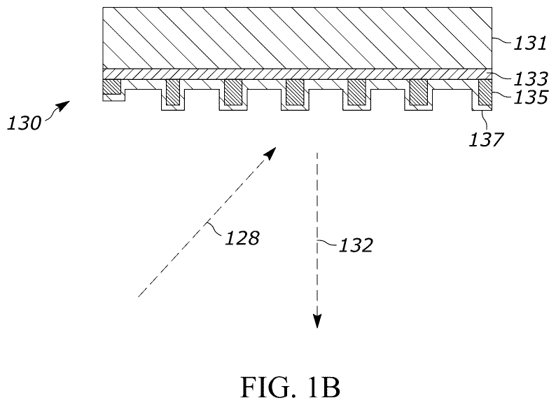

FIGS. 1A-1B are diagrams illustrating an optical system 100 with which various embodiments can be practiced. More specifically, FIG. 1A is a block diagram illustrating the optical system 100 according to one example. FIG. 1B is a diagram illustrating a cross-sectional side view of a biosensor 130 that is interrogated with the optical system 100 according to one example.

In the example shown, the optical system 100 includes a light source 110, an excitation filter 120, lenses 124, 140, and 156, mirrors 126, 154, and 158, an emission filter 150, a spectrometer 160, a pixelated optical detector (e.g., a CCD) 170, and a computing device 180. In operation, the light source 110 generates an output light beam 112, which is directed through the excitation filter 120 and the focusing lens 124 toward the mirror 126. The mirror 126 redirects a received filtered excitation light beam 128 toward the biosensor 130. In response to the excitation light beam 128, the biosensor 130 generates an emission (including fluorescence) light beam 132, which is collected by the lens 140, passes through the emission filter 150 and is relayed by the mirrors 154 and 158 and the lens 156 to the spectrometer 160. The spectrometer 160 disperses the received light in wavelength, and the spectrally dispersed light is detected with the pixelated optical detector 170. A readout signal 172 from the pixelated optical detector 170 is directed to the computing device 180 for processing.

In various examples, the excitation filter 120 is a fixed low-pass filter, a fixed bandpass filter, or a variable bandpass filter. In some examples, one or both of the light source 110 and the excitation filter 120 are tunable, which enables the optical system 100 to change the spectral composition of the excitation light beam 128. In some examples, the light source 110 and the excitation filter 120 are configured to make the excitation light beam 128 substantially monochromatic or substantially dichromatic. In some examples, the excitation light beam 128 has a wavelength in the UV range, such as the wavelength of about 266 nm.

In various examples, the emission filter 150 is a fixed long-pass filter or a variable long-pass filter. In some examples, the emission filter 150 is absent, and the spectrometer 160 performs the rejection of unwanted wavelengths instead of the emission filter 150.

Referring to FIG. 1B, in the example shown, the biosensor 130 includes a silicon substrate layer 131 with a thin (˜1.7 nm) native silicon-oxide layer 133. The biosensor 130 also includes a profiled Al layer 135 adjacent to and bonded to the silicon-oxide layer 133. The profiled Al layer 135 has an approximately periodic array of features of varying thickness with a maximum thickness being approximately 30 nm. The profiled Al layer 135 also typically has a relatively thin (˜4 nm) native aluminum oxide layer at the exterior surface thereof. The biosensor 130 has a relatively thin sample layer 137 deposited onto the exterior surface of the profiled Al layer 135 and comprising polyvinyl alcohol (PVA) and neurotransmitter molecules. In one example, the sample layer 137 is produced by dissolving the neurotransmitter molecules in a PVA solution and then using the resulting solution to spin-coat the exterior surface of the profiled Al layer 135.

In one example, the biosensor 130 illustrated in FIG. 1B can be fabricated as follows. First, polystyrene (PS) nanospheres are spin-coated for 70 seconds at 800 rpm onto the wafer 131/133. Then, PS is etched on the wafer (e.g., using the Oxford Plasmalab 80 Plus) until the nanospheres reach the diameter of 216 (+6) nm. Following the etching process, a 30 nm-thick layer of Al is deposited over the etched PS nanospheres by electron beam evaporation. The residual PS particles are removed by ultrasonication in isopropyl alcohol (IPA) for one hour. As a result, the Al hole array layer 135 with hole sizes of 215 (+5) nm and hole spacing of 300 nm is formed. Neurotransmitter molecules are dissolved in a 0.25 wt. % PVA solution, which is then spin-coated for 30 seconds at 3000 rpm on the surface of the Al hole array layer 135. The thickness of the PVA film containing the neurotransmitter molecules after solvent evaporation is about 10 nm.

In other examples, other suitable fabrication methods can also be used to fabricate a plasmonic-engineered layer 135 on the wafer 131/133. For example, some of such methods can be designed by borrowing certain elements of lithographic chip fabrication techniques used in the microelectronics industry. Such techniques can beneficially be adapted to produce substantially any desired topology in the plasmonic-engineered layer 135.

In one example, the optical system 100 can be used to measure the native fluorescence of neurotransmitters on a substrate as follows. A 12-mW optical power output beam 112 generated by the high-intensity 266-nm UV continuous-wave (CW) laser 110 is focused by the plano-convex lens 124 (with the focal length of 100 mm) to a spot size of 60 μm by 60 μm on the sample layer 137 at a 60-degree incident angle. The native fluorescence emission 132 of neurotransmitters is excited in the illuminated spot and is collected by the UV objective 140 (with the focal length of 25 mm, F/2.8). The fluorescence emission 132 passes through the long-pass filter 150 and the cylindrical lens 156 (with the focal length of 100 mm) and enters into the imaging spectrometer 160 coupled to the UV-enhanced CCD camera 170. A photobleaching series of fluorescence spectra is collected by the computing device 180 by operating the CCD camera 180 using a 0.5-second integration time for a total time duration of 90 seconds.

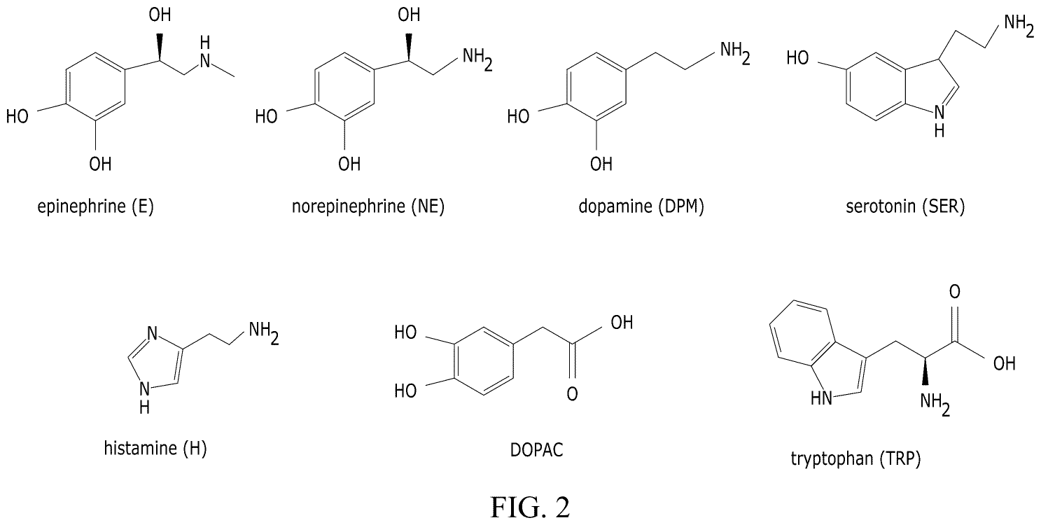

FIG. 2 schematically illustrates the chemical structures of several neurotransmitter molecules that can be included in the sample layer 137 on the biosensor 130 according to some examples. Neurotransmitters are essential chemicals that play a crucial role in the communication between neurons in the brain and other parts of the nervous system. For example, neurotransmitters are responsible for transmitting information between nerve cells, allowing them to communicate and coordinate different functions throughout the body. Without neurotransmitters, the brain and nervous system would not be able to function properly. There are dozens of neurotransmitters that have been identified, each with its own unique function and mode of action. For illustration purposes and without any implied limitations, FIG. 2 shows only some neurotransmitters. In various additional examples, other neurotransmitter molecules may similarly be included in the sample layer 137 on the biosensor 130.

Dopamine (DA) is perhaps the most well-known neurotransmitter, as it is often associated with pleasure and reward. DA is involved in regulating movement, motivation, attention, and learning. Too much or too little dopamine can lead to a range of disorders, such as the Parkinson's disease, schizophrenia, and addiction. 3,4-Dihydroxyphenylacetic acid (DOPAC) is a metabolite of DA.

Serotonin (SER) is another important neurotransmitter that is involved in regulating mood, appetite, and sleep. SER is sometimes referred to as the “feel-good” neurotransmitter, as it can induce feelings of happiness and well-being. Low SER levels are linked to depression, anxiety, and eating disorders. L-tryptophan (TRP) is the precursor of serotonin.

Norepinephrine (NE) is a neurotransmitter that is involved in the body's “fight or flight” response. NE helps to regulate heart rate, blood pressure, and breathing during stressful situations. Low levels of norepinephrine are linked to depression and anxiety.

Epinephrine (E), also known as adrenaline, is both a neurotransmitter and a hormone. It plays an important role in the body's “fight or flight” response. Epinephrine is also used as a medication to treat many life-threatening conditions.

Histamine (H) is a monoamine neurotransmitter that is synthesized from histidine via l-histidine decarboxylase (HDC). Histamine is released by the immune system and is mainly known for its role in causing allergy symptoms. Other important functions of histamine include regulating the body's “sleep-wake” cycle and cognitive function. Antihistamines are common medications that can be used to manage histamine levels.

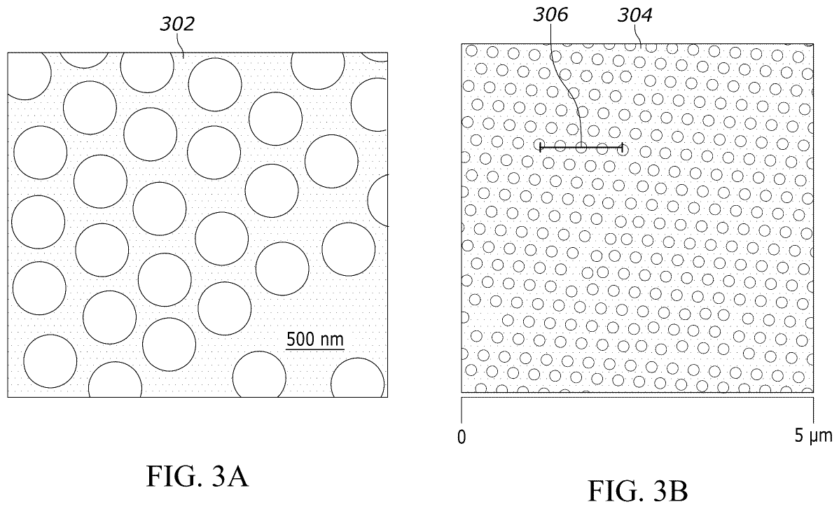

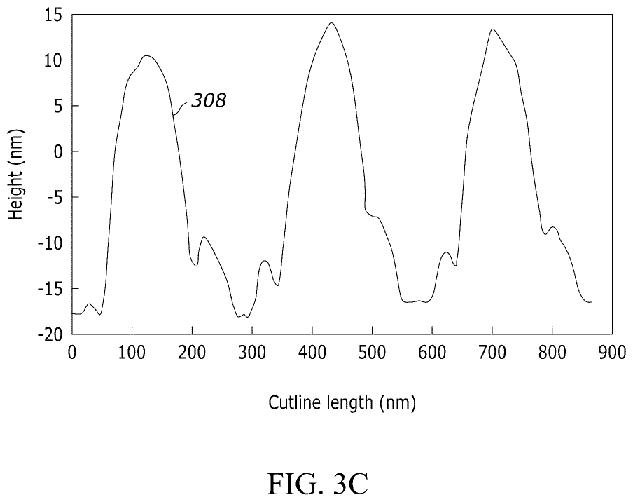

FIGS. 3A-3C pictorially and graphically illustrate the biosensor 130 according to one example. More specifically, FIGS. 3A and 3B show schematic outlines 302 and 304 of a scanning electron microscope (SEM) image and an atomic force microscope (AFM) image, respectively, of the biosensor 130 prior to the deposition of the sample layer 137. In the example shown, the profiled Al layer 135 of the biosensor 130 has an array of substantially spherical-dome-shaped holes with an approximately 300 nm center-to-center hole spacing. FIG. 3C graphically shows a feature height profile 308 along a cutline 306 indicated in the AFM image 304 (FIG. 3B). Herein below, the profiled Al layer 135 illustrated in FIGS. 3A-3C may be referred to as the “P300 Al hole array” layer 135.

For some applications, different embodiments of the biosensor 130 can be engineered by selecting and/or changing one or more of the following characteristics thereof: (i) the plasmonic material (e.g., Al, Au, Ag, Cu, Ti, Cr, TiN, ZrN, HIN, VN, NbN, etc.) used in the profiled layer 135; (ii) the amplitude of the feature height variation across the profiled layer 135 (e.g., analogous to the amplitude of the feature height profile 308, FIG. 3C); (iii) the transverse (in-layer) size of the holes in the profiled layer 135; (iv) the average distance between the holes in the profiled layer 135; and (v) the geometric shape(s) (e.g., cylindrical, hemispherical, spherical-dome, spherical-segment, conical, etc.) of the holes in the profiled layer 135. In some embodiments, the biosensor 130 may have a flat (constant-thickness) layer 135 of a selected plasmonic material in place of the profiled layer 135.

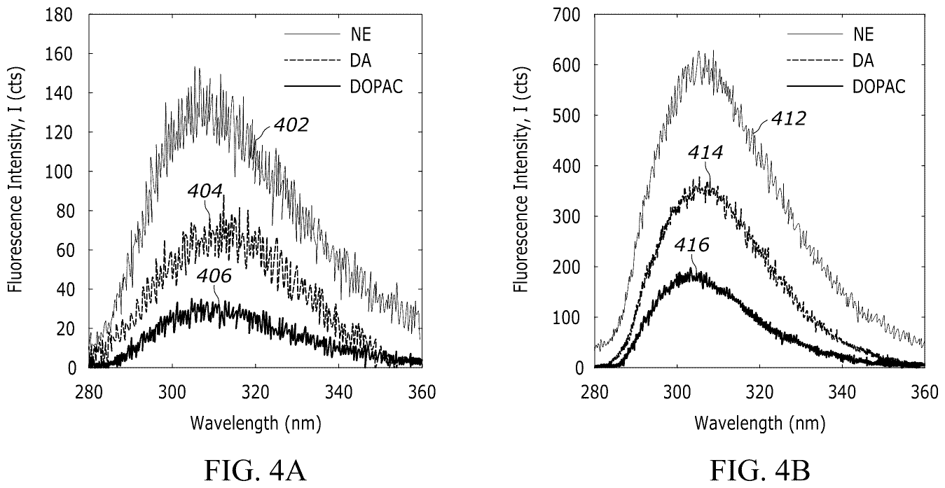

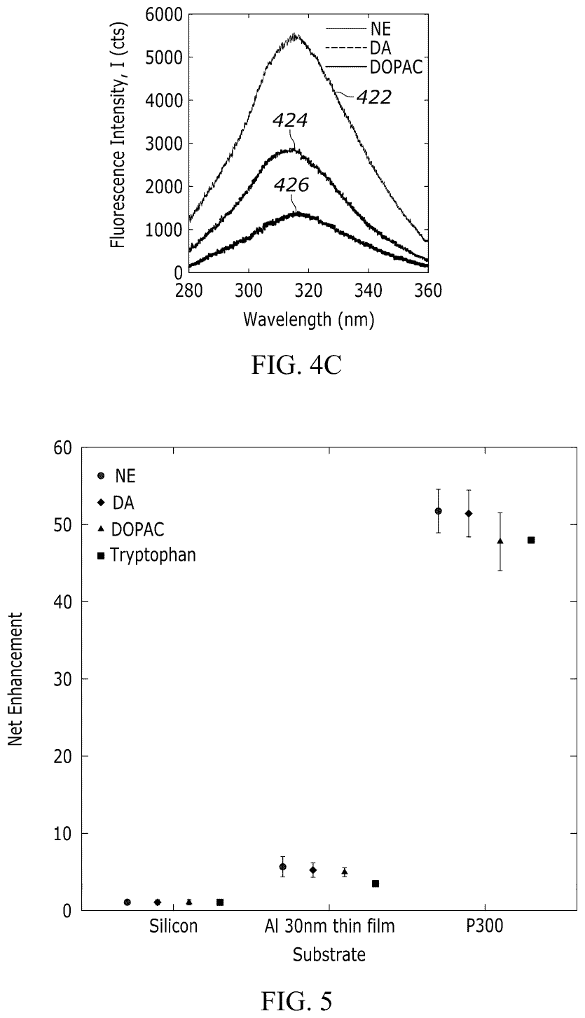

FIGS. 4A-4C graphically illustrate emission spectra of several neurotransmitters obtained with the optical system 100 according to some examples. The experimental data shown FIGS. 4A-4C correspond to different biosensors 130 in which the sample layer 137 is deposited onto the bare silicon/silicon-oxide wafer 131/133, the flat 30-nm thick Al layer 135 on the silicon/silicon-oxide wafer 131/133, and the P300 Al hole array layer 135 illustrated in FIG. 3, respectively. Spectra 402, 412, 422 correspond to the sample layers 137 containing NE. Spectra 404, 414, 424 correspond to the sample layers 137 containing DA. Spectra 406, 416, 426 correspond to the sample layers 137 containing DOPAC.

All fluorescence spectra shown in FIGS. 4A-4C exhibit an emission maximum spectrally located between 300 nm and 320 nm. The spectral peak locations for the neurotransmitter molecules on the P300 Al hole array layer 135 (FIG. 4C) are red shifted compared to those on the bare silicon/silicon-oxide wafer 131/133 (FIG. 4A) and the flat 30-nm thick Al layer (FIG. 4B). This red shift can be attributed to the change in the local refractive index on the Al hole array substrate. The emission intensity is the highest for NE (see the spectra 402, 412, 422), followed by DA (see the spectra 404, 414, 424) and DOPAC (see the spectra 406, 416, 426), on each of the three substrates, which is also consistent with the relative emission intensities observed in the bulk solution. The type of substrate may affect the emission intensity relatively strongly. For example, the emission intensity on the P300 Al hole array layer 135 (FIG. 4C) is the highest, followed by that on the flat 30-nm thick Al layer (FIG. 4B) and the bare silicon/silicon-oxide wafer 131/133 (FIG. 4A). The significant observed fluorescence-signal enhancement on the P300 Al hole array layer 135 (FIG. 4C) compared to the other two substrates is due to the surface plasmon resonance phenomenon exhibited by the P300 Al hole array layer 135 at the excitation wavelength of 266 nm.

FIG. 5 graphically illustrates the net native fluorescence enhancement for several neurotransmitter molecules achieved with different biosensors 130 according to some examples. More specifically, the three biosensors 130 represented in FIG. 5 are the above-described biosensors in which the sample layer 137 is deposited onto the bare silicon/silicon-oxide wafer 131/133, the flat 30-nm thick Al layer 135 on the silicon/silicon-oxide wafer 131/133, and the P300 Al hole array layer 135 illustrated in FIG. 3, respectively. The native-fluorescence level observed on the bare silicon/silicon-oxide wafer 131/133 is taken as the reference level of one unit on the enhancement scale for each of the indicated molecules. The flat 30-nm thick Al layer 135 on the silicon/silicon-oxide wafer 131/133 provides the native-fluorescence enhancement factor of approximately five. The P300 Al hole array layer 135 provides the native-fluorescence enhancement factor of approximately fifty.

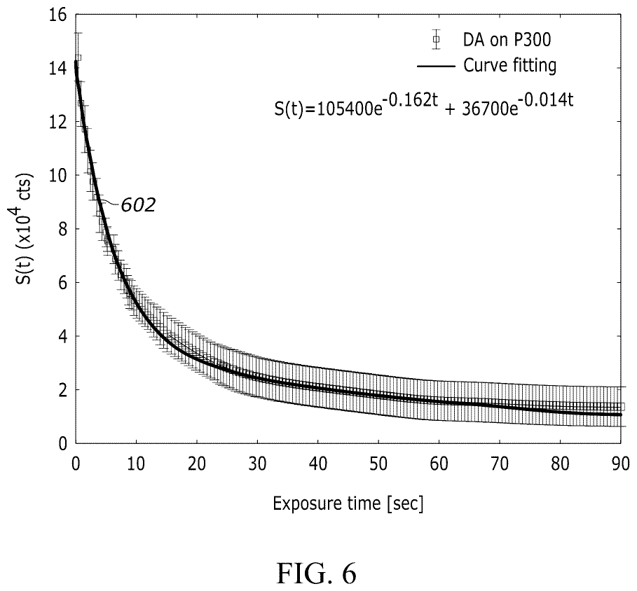

FIG. 6 graphically shows a kinetic curve 602 of native-fluorescence decay observed for dopamine (DA) in the sample layer 137 on the P300 Al hole array layer 135 of the biosensor 130 according to one example. In other examples, similar kinetic curves are measured with the optical system 100 for different individual neurotransmitters on different types of biosensor 130. The native fluorescence typically completely bleaches under the used illumination conditions down to the background noise after approximately 90 seconds.

In one example, to obtain the kinetic curve 602, the fluorescence decay series is measured for 90 seconds continuously with 0.5-second integration time per spectrum. A typical series is characterized in that the fluorescence intensity falls over time while the peak positions remain stable (substantially unchanged). We denote the measured fluorescence intensity as I(λ,t), i.e., the fluorescence intensity is wavelength- and time-dependent. For the determination of photobleaching rates, we obtain S(t), which represents the integrated fluorescence intensity over the wavelength range from 280 nm to 360 nm expressed as follows:

$$S ( t ) = {\int }_{280}^{360} I ( λ , t ) d λ \qquad ( 1 )$$

The photobleaching rates are obtained by fitting the integrated fluorescence intensity S(t) with the following two-term exponential function:

$$S ( t ) = a \times exp ( - {k}_{1} t ) + b \times exp ( - {k}_{2} t ) \qquad ( 2 )$$

where a and b are amplitudes. The first exponential term in Eq. (2) represents the fast decay with the rate constant k₁, and the second exponential term in Eq. (2) represents the slow decay with the rate constant k₂. An example fit of the kinetic curve 602 with Eq. (2) is shown in FIG. 6 along with the best-fit parameter values for a, b, k₁, and k₂.

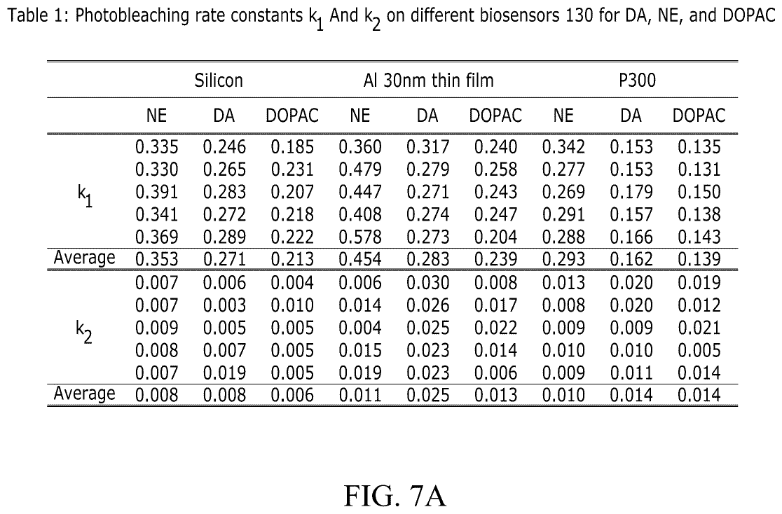

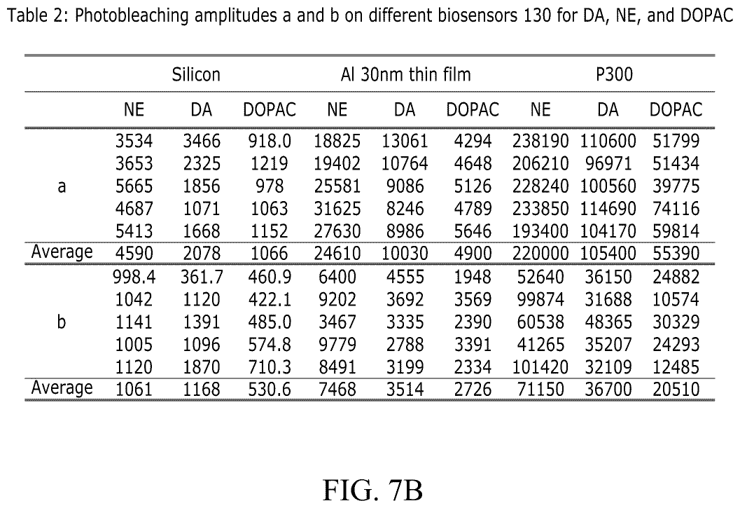

Herein, the term “photobleaching” refers to photochemical alteration of a fluorophore molecule that makes it permanently unable to fluoresce because some portions thereof are broken down by photochemical reactions. Irreversible photobleaching of neurotransmitters is observed on biosensors 130, e.g., as indicated by the data shown in FIG. 6. The photobleaching rate(s) can be obtained as described above and then used to characterize the corresponding analytes according to various embodiments described in more detail below in reference to FIGS. 7-10.

FIGS. 7A and 7B are tables illustrating sets of photobleaching parameters for several neurotransmitter molecules on several different biosensors 130 according to some examples. More specifically, the table shown in FIG. 7A lists photobleaching rate constants k₁ and k₂ on the indicated biosensors 130 for DA, NE, and DOPAC. The table shown in FIG. 7B similarly lists photobleaching amplitudes a and b on the indicated biosensors 130 for DA, NE, and DOPAC.

Each of the photobleaching parameters shown in the tables of FIGS. 7A-7B is calculated as an average taken over five measurements performed with nominally identical samples on nominally identical biosensors 130. The first photobleaching rate constant k₁ is at least three times larger than the second photobleaching rate constants k₂, as evident from Table 1. For the three shown neurotransmitter molecules, NE has the largest k₁ on the three types of substrates, which is followed by DA and DOPAC. In other words, DOPAC is the most photostable molecule, followed by DA and NE.

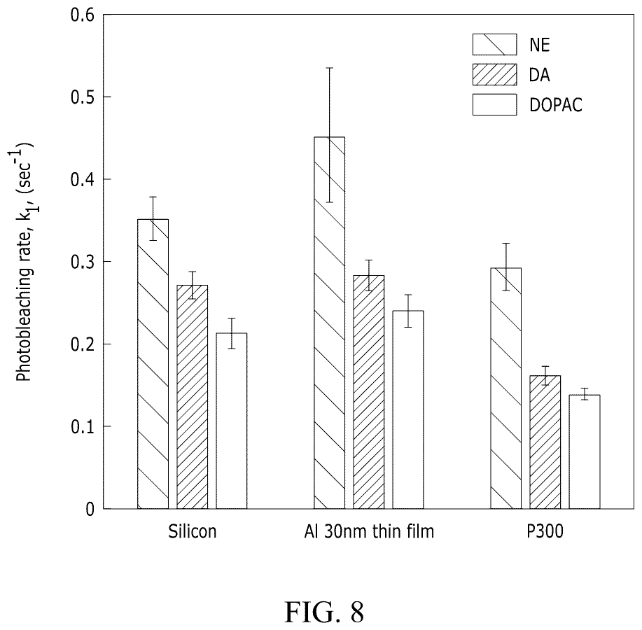

FIG. 8 graphically compares the first photobleaching rate constants k₁ of several neurotransmitter molecules on several different biosensors 130 according to some examples. Also shown in FIG. 8 are the error bars representing the standard deviations of the measured values determined based on the five duplicative measurements indicated in Table 1 (FIG. 7A). The data shown in FIG. 8 clearly indicate that the differences in k₁ values among the indicated neurotransmitter molecules are larger than the standard deviations. As such, the measured photobleaching rates of native fluorescence can be used to distinguish different neurotransmitter molecules based at least on their respective distinct values of the first photobleaching rate constant k₁.

In other examples, photobleaching parameters for other pertinent neurotransmitter molecules on different biosensors 130 are similarly processed and tabulated. In some examples, the processing operations may include normalization, standardization, and other suitable data preprocessing. The resulting tables of photobleaching parameters provide calibration data that are used with the optical system 100 in at least some embodiments. In some examples, the photobleaching calibration data are converted into one or more corresponding lookup tables (LUTs) and stored in a memory accessible to or from the computing device 180. For example, upon performing an analysis of the photobleaching kinetics acquired with the optical system 100 for an analyte under test (AUT), the computing device 180 can query the corresponding LUT(s) and then provide certain conclusions or predictions with respect to that AUT based on the response to the query returned from the LUT(s).

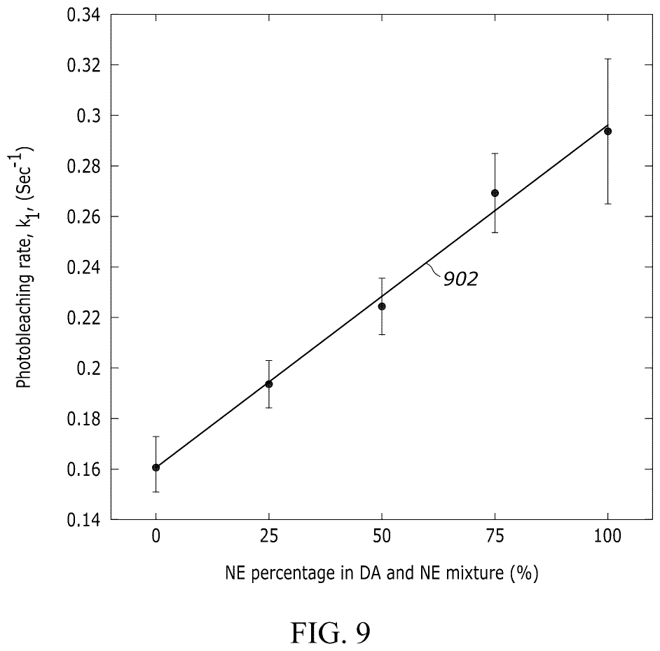

FIG. 9 graphically illustrates the relative-concentration dependence of the first photobleaching rate constant k₁ for the sample layer 137 of the biosensor 130 containing a mixture of two neurotransmitters according to one example. In the example shown, the two neurotransmitters are DA and NE. The used biosensor 130 includes the above-described P300 Al hole array layer 135. Different experimental points shown in FIG. 9 correspond to different DA and NE mixtures prepared in volume ratios of 1:0, 3:1, 1:1, 1:3, and 0:1 (which correspond to 100%, 75%, 50%, 25%, and 0%, respectively, of DA in the mixture). A straight line 902 represents the best fit to the shown set of experimental points obtained via the linear regression analysis. The data shown in FIG. 9 clearly indicate that the effective value of the first photobleaching rate constant k₁ changes linearly with the relative percentage of the two neurotransmitters in the mixture. As such, the measured photobleaching rate of native fluorescence can be used to determine the relative concentration of two known neurotransmitters in the mixture having an unknown relative concentration.

In other examples, the linear regression parameters for other neurotransmitter pairs on different biosensors 130 are similarly obtained, processed, and tabulated. In some examples, the processing operations may include normalization, standardization, and other suitable preprocessing functions. The resulting tables provide calibration data that are used with the optical system 100 in at least some embodiments. In some examples, the calibration data are converted into one or more corresponding LUTs and stored in a memory accessible to or from the computing device 180. For example, upon performing an analysis of the photobleaching kinetics acquired with the optical system 100 for an AUT containing an unknown ratio of two known neurotransmitters, the computing device 180 can query the corresponding LUT(s) to obtain an accurate estimate of that ratio in the AUT based on the response to the query returned from the LUT(s).

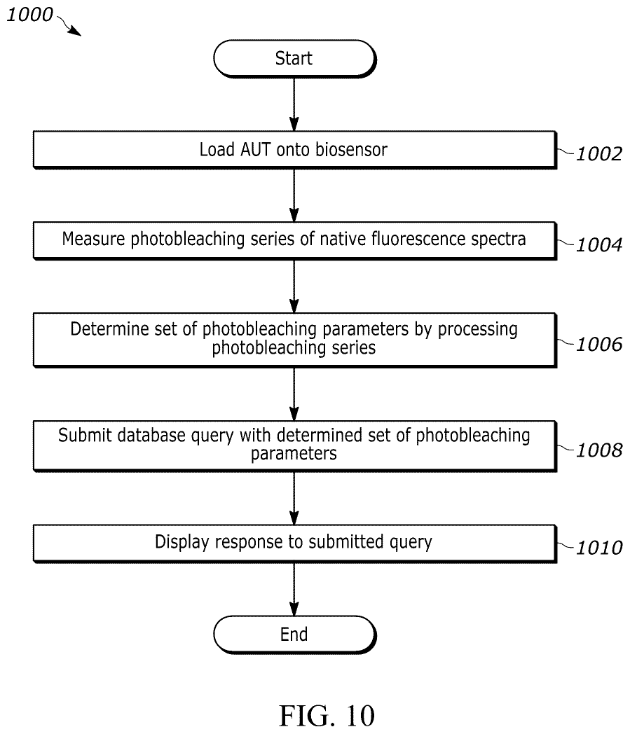

FIG. 10 is a flowchart illustrating a method 1000 of detecting and differentiating neurotransmitters according to some examples. The method 1000 can be implemented using the optical system 100 and further using appropriate software installed on the computing device 180 and/or a remote network-connected server. The method 1000 is described below with continued reference to FIGS. 1-10.

A block 1002 of the method 1000 includes loading an AUT onto a biosensor 130. In some examples, operations of the block 1002 include selecting the biosensor 130 from a plurality of different biosensors 130. As indicated above in reference to FIGS. 3A-3C, different biosensors 130 of the plurality may differ in one or more of the following characteristics: (i) the plasmonic material used in the layer 135; (ii) the amplitude of the feature height variation across the layer 135; (iii) the transverse (in-layer) size of the holes in the layer 135; (iv) the average distance between the holes in the layer 135; (v) the geometric shapes of the holes in the layer 135, etc. One of the available biosensors 130 may be selected in the block 1002 based on auxiliary information about the AUT. For example, when the neurotransmitter concentration(s) in the AUT is (are) expected to be relatively low, a biosensor 130 providing a relatively high level of signal enhancement may be selected. In another example, when the relative concentrations of two known neurotransmitters in the AUT need to be determined, a biosensor 130 providing an approximately largest difference in one or more pertinent signal characteristics for the two neurotransmitters may be selected. In yet some other examples, other considerations (such as various trial and error approaches) for selecting the biosensor 130 in the block 1002 can also be employed.

The operations of the block 1002 further include forming the sample layer 137 on the selected biosensor 130 using the AUT. In some examples, the spin-coating technique described above in reference to FIG. 1B may be used for this purpose. In some other examples, other suitable techniques for forming the AUT-containing sample layer 137 on the selected biosensor 130 can similarly be employed in the block 1002.

A block 1004 of the method 1000 includes the computing device 180 (serving as an electronic controller of the optical system 100) controlling various system components to measure a photobleaching series of native fluorescence spectra using the loaded biosensor 130 prepared in the block 1002. In some examples, the measurements of the block 1004 are performed as described above in reference to FIGS. 1A-1B. In such examples, the excitation light at 266 nm serves a dual purpose of both exciting the native fluorescence in the AUT and inducing the photochemical reactions that alter the neurotransmitter molecules in the AUT thereby making them unable to fluoresce and quenching the observed native fluorescence, e.g., as illustrated by the kinetic curve 602 (FIG. 6). In some other examples, the fluorescence measurements in the block 1004 are implemented such that a first wavelength is used to excite the native fluorescence whereas a different second wavelength is used to induce the photochemical reactions. In such examples, the computing device 180 configures the light source 110 and/or the excitation filter 120 to switch the excitation light beam 128 between the first and second wavelengths during the acquisition of the photobleaching series as needed.

A block 1006 of the method 1000 includes the computing device 180 processing the photobleaching series of native fluorescence spectra measured in the block 1004 to determine a corresponding set of photobleaching parameters. In some examples, the processing operations include (i) integrating the fluorescence intensity in accordance with Eq. (1) and (ii) fitting the obtained values of S(t) with the two-term exponential function of Eq. (2). The corresponding set of parameters obtained form the fit include the rate constants k₁, and k₂ and the amplitudes a and b. In some other examples, other suitable mathematical models and/or parameterization of the photobleaching kinetics can also be used.

A block 1008 of the method 1000 includes the computing device 180 submitting a query to a database with the set of photobleaching parameters determined in the block 1006. In one example, the database includes a plurality of LUTs having stored therein various photobleaching calibration data. The plurality of LUTs includes a first set of calibration LUTs having stored therein the photobleaching calibration data exemplified by Table 1 and Table 2 (FIGS. 7A-7B). The plurality of LUTs also includes a second set of calibration LUTs having stored therein the photobleaching calibration data exemplified by FIG. 9. In some examples, the plurality of LUTs may also include one or more additional sets of calibration LUTs having stored therein additional photobleaching calibration data. In different examples, the database may be stored locally in the computing device 180 or be stored in a remote server that is network-connected to the computing device 180.

In some examples, the query submitted in the block 1008 is configured based on the user input and contains a numerical field specifying the query type. The query type determines which set of calibration LUTs the query will be directed to in the database. For example, when the query type is set to “type=0,” the query will be routed in the database to the above-described first set of calibration LUTs. When the query type is set to “type=1,” the query will be routed in the database to the above-described second set of calibration LUTs, and so on. The query submitted in the block 1008 also contains at least a subset of the set of photobleaching parameters determined in the block 1006 and the identifier of the biosensor 130 selected in the block 1002. In various examples, the query submitted in the block 1008 also typically contains any applicable auxiliary information about the AUT and other pertinent metadata.

A block 1010 of the method 1000 includes the computing device 180 receiving a response to the query submitted in the block 1008 and optionally displaying for the user the received response on a display device. The contents of the received response typically depend on the query type. For example, a response to the query “type=0” may contain a predicted identity of the neurotransmitter in the AUT and its estimated concentration or amount in the sample layer 137 of the biosensor 130. The neurotransmitter identity can be predicted by matching one or both of the rate constants k₁, and k₂ to the corresponding data in the first set of calibration LUTs. The concentration or amount of the neurotransmitter in the sample layer 137 can be estimated based on one or both of the amplitudes a and b. In some examples, the received response contains a ranked list of predicted identities along with their confidence scores computed based on the “distances” between the set of photobleaching parameters determined in the block 1006 and the corresponding calibration sets of photobleaching parameters. As another example, a response to the query “type=1” may contain a percentage ratio of the two neurotransmitters in the AUT determined based on the photobleaching calibration data stored in the second set of calibration LUTs. Upon completion of the operations of the block 1010, the processing of the method 1000 is terminated.

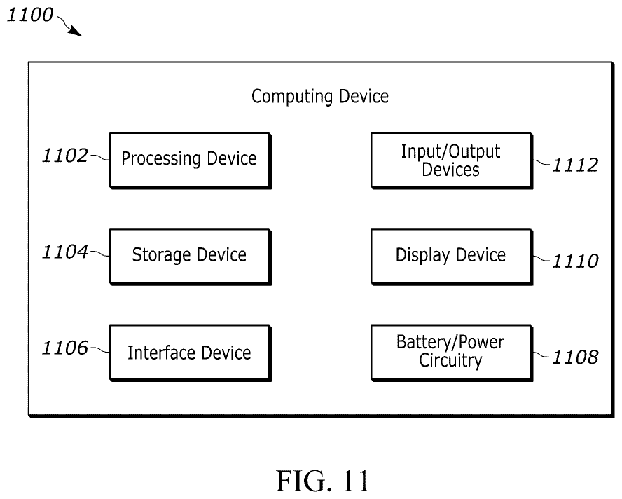

FIG. 11 is a block diagram illustrating a computing device 1100 used in the optical system 100 according to some examples. In various examples, the optical system 100 may include a single computing device 1100 or multiple computing devices 1100. In some examples, the computing device 1100 implements the computing device 180 (also see FIG. 1). In various examples, an instance of the computing device 1100 can be used to implement the method 1000.

The computing device 1100 of FIG. 11 is illustrated as having a number of components, but any one or more of these components may be omitted or duplicated, as suitable for the application and setting. In some embodiments, some or all of the components included in the computing device 1100 may be attached to one or more motherboards and enclosed in a housing. In some embodiments, some of those components may be fabricated onto a single system-on-a-chip (SoC) (e.g., the SoC may include one or more electronic processing devices 1102 and one or more storage devices 1104). Additionally, in various embodiments, the computing device 1100 may not include one or more of the components illustrated in FIG. 11, but may include interface circuitry for coupling to the one or more components using any suitable interface (e.g., a Universal Serial Bus (USB) interface, a High-Definition Multimedia Interface (HDMI) interface, a Controller Area Network (CAN) interface, a Serial Peripheral Interface (SPI) interface, an Ethernet interface, a wireless interface, or any other appropriate interface). For example, the computing device 1100 may not include a display device 1110, but may include display device interface circuitry (e.g., a connector and driver circuitry) to which an external display device 1110 may be coupled.

The computing device 1100 includes a processing device 1102 (e.g., one or more processing devices). As used herein, the terms “electronic processor device” and “processing device” interchangeably refer to any device or portion of a device that processes electronic data from registers and/or memory to transform that electronic data into other electronic data that may be stored in registers and/or memory. In various embodiments, the processing device 1102 may include one or more digital signal processors (DSPs), application-specific integrated circuits (ASICs), central processing units (CPUs), graphics processing units (GPUs), server processors, field programmable gate arrays (FPGA), or any other suitable processing devices.

The computing device 1100 also includes a storage device 1104 (e.g., one or more storage devices). In various embodiments, the storage device 1104 may include one or more memory devices, such as random-access memory (RAM) devices (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some embodiments, the storage device 1104 may include memory that shares a die with the processing device 1102. In such an embodiment, the memory may be used as cache memory and include embedded dynamic random-access memory (eDRAM) or spin transfer torque magnetic random-access memory (STT-MRAM), for example. In some embodiments, the storage device 1104 may include non-transitory computer readable media having instructions thereon that, when executed by one or more processing devices (e.g., the processing device 1102), cause the computing device 1100 to perform any appropriate ones of the methods disclosed herein below or portions of such methods.

The computing device 1100 further includes an interface device 1106 (e.g., one or more interface devices 1106). In various embodiments, the interface device 1106 may include one or more communication chips, connectors, and/or other hardware and software to govern communications between the computing device 1100 and other computing devices. For example, the interface device 1106 may include circuitry for managing wireless communications for the transfer of data to and from the computing device 1100. The term “wireless” and its derivatives may be used to describe circuits, devices, systems, methods, techniques, communications channels, etc., that may communicate data via modulated electromagnetic radiation through a nonsolid medium. The term does not imply that the associated devices do not contain any wires, although in some embodiments they might not. Circuitry included in the interface device 1106 for managing wireless communications may implement any of a number of wireless standards or protocols, including but not limited to Institute for Electrical and Electronic Engineers (IEEE) standards including Wi-Fi (IEEE 802.11 family), IEEE 802.16 standards, Long-Term Evolution (LTE) project along with any amendments, updates, and/or revisions (e.g., advanced LTE project, ultramobile broadband (UMB) project (also referred to as “3GPP2”), etc.). In some embodiments, circuitry included in the interface device 1106 for managing wireless communications may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. In some embodiments, circuitry included in the interface device 1106 for managing wireless communications may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). In some embodiments, circuitry included in the interface device 1106 for managing wireless communications may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some embodiments, the interface device 1106 may include one or more antennas (e.g., one or more antenna arrays) configured to receive and/or transmit wireless signals.

In some embodiments, the interface device 1106 may include circuitry for managing wired communications, such as electrical, optical, or any other suitable communication protocols. For example, the interface device 1106 may include circuitry to support communications in accordance with Ethernet technologies. In some embodiments, the interface device 1106 may support both wireless and wired communication, and/or may support multiple wired communication protocols and/or multiple wireless communication protocols. For example, a first set of circuitry of the interface device 1106 may be dedicated to shorter-range wireless communications such as Wi-Fi or Bluetooth, and a second set of circuitry of the interface device 1106 may be dedicated to longer-range wireless communications such as global positioning system (GPS), EDGE, GPRS, CDMA, WiMAX, LTE, EV-DO, or others. In some other embodiments, a first set of circuitry of the interface device 1106 may be dedicated to wireless communications, and a second set of circuitry of the interface device 1106 may be dedicated to wired communications.

The computing device 1100 also includes battery/power circuitry 1108. In various embodiments, the battery/power circuitry 1108 may include one or more energy storage devices (e.g., batteries or capacitors) and/or circuitry for coupling components of the computing device 1100 to an energy source separate from the computing device 1100 (e.g., to AC line power).

The computing device 1100 also includes a display device 1110 (e.g., one or multiple individual display devices). In various embodiments, the display device 1110 may include any visual indicators, such as a heads-up display, a computer monitor, a projector, a touchscreen display, a liquid crystal display (LCD), a light-emitting diode display, or a flat panel display.

The computing device 1100 also includes additional input/output (I/O) devices 1112. In various embodiments, the I/O devices 1112 may include one or more data/signal transfer interfaces, audio I/O devices (e.g., microphones or microphone arrays, speakers, headsets, earbuds, alarms, etc.), audio codecs, video codecs, printers, sensors (e.g., thermocouples or other temperature sensors, humidity sensors, pressure sensors, vibration sensors, etc.), image capture devices (e.g., one or more cameras), human interface devices (e.g., keyboards, cursor control devices, such as a mouse, a stylus, a trackball, or a touchpad), etc.

Depending on the specific embodiment of the optical system 100, various components of the interface devices 1106 and/or I/O devices 1112 can be configured to send and receive suitable control messages, suitable control/telemetry signals, and streams of data. In some examples, the interface devices 1106 and/or I/O devices 1112 include one or more analog-to-digital converters (ADCs) for transforming received analog signals into a digital form suitable for operations performed by the processing device 1102 and/or the storage device 1104. In some additional examples, the interface devices 1106 and/or I/O devices 1112 include one or more digital-to-analog converters (DACs) for transforming digital signals provided by the processing device 1102 and/or the storage device 1104 into an analog form suitable for being communicated to the corresponding components of the optical system 100.

According to an example embodiment disclosed above, e.g., in the summary section and/or in reference to any one or any combination of some or all of FIGS. 1-11, provided is an apparatus comprising: a fluorimeter configured to measure a time series of fluorescence spectra of a biosensor having loaded thereon an analyte and subjected to illumination by ultraviolet light, the biosensor including an engineered layer of plasmonic material, the analyte including a neurotransmitter, different ones of the fluorescence spectra in the time series corresponding to different respective illumination times; and a computing device configured to: determine a first rate constant based on the time series, the first rate constant corresponding to a photochemical reaction of the neurotransmitter caused by the ultraviolet light; submit to a database a query containing at least the determined first rate constant, the database including photobleaching calibration data representing a plurality of different neurotransmitters and a plurality of different biosensors; and display on a display device a response to the submitted query received from the database.

In some embodiments of the above apparatus, the computing device is further configured to: fit an exponential function to fluorescence decay kinetics represented by the time series; and determine the first rate constant based on the fit.

In some embodiments of any of the above apparatus, the computing device is further configured to: integrate over a range of wavelengths each of the fluorescence spectra in the time series; and obtain the fit using the integrated fluorescence spectra.

In some embodiments of any of the above apparatus, the exponential function has first and second exponential components; wherein the first rate constant corresponds to the first exponential component; wherein the computing device is further configured to determine, based on the fit, a second rate constant corresponding to the second exponential component, a first amplitude corresponding to the first exponential component, and a second amplitude corresponding to the second exponential component; and wherein the query further contains at least one of the second rate constant, the first amplitude, and the second amplitude.

According to another example embodiment disclosed above, e.g., in the summary section and/or in reference to any one or any combination of some or all of FIGS. 1-11, provided is a method comprising: determining a first rate constant based on a time series of fluorescence spectra of a biosensor having loaded thereon an analyte and subjected to illumination by ultraviolet light, the biosensor including an engineered layer of plasmonic material, the analyte including a neurotransmitter, the first rate constant corresponding to a photochemical reaction of the neurotransmitter caused by the ultraviolet light; submitting to a database a query containing at least the determined first rate constant, the database including photobleaching calibration data representing a plurality of different neurotransmitters and a plurality of different biosensors; and receiving from the database a response to the submitted query.

In some embodiments of the above method, the determining comprises: fitting an exponential function to fluorescence decay kinetics represented by the time series; and determining the first rate constant based on the fitting.

In some embodiments of any of the above methods, the determining further comprises: integrating over a range of wavelengths each of the fluorescence spectra; and performing the fitting using the integrated fluorescence spectra.

In some embodiments of any of the above methods, the exponential function has first and second exponential components; wherein the first rate constant corresponds to the first exponential component; and wherein the determining further comprises determining, based on the fitting, a second rate constant corresponding to the second exponential component, a first amplitude corresponding to the first exponential component, and a second amplitude corresponding to the second exponential component.

In some embodiments of any of the above methods, the query further contains at least one of the second rate constant, the first amplitude, and the second amplitude.

In some embodiments of any of the above methods, the neurotransmitter is selected from the group consisting of: a monoamine neurotransmitter (MANT); dopamine (DA); serotonin (SER); norepinephrine (NE); epinephrine (E); histamine (H); tryptophan (TRP); and 3,4-dihydroxyphenylacetic acid (DOPAC).

In some embodiments of any of the above methods, the plasmonic material comprises a material selected from the group consisting of Al, Au, Ag, Cu, Ti, Cr, TiN, ZrN, HIN, VN, and NbN.

In some embodiments of any of the above methods, the ultraviolet light causes both the fluorescence and the photochemical reaction.

In some embodiments of any of the above methods, the engineered layer has a varying feature height across the biosensor.

In some embodiments of any of the above methods, the response includes a characteristic selected from the group consisting of: a predicted identity of the neurotransmitter; a list of predicted identities of the neurotransmitter ranked based on respective confidence scores; an estimated amount of the neurotransmitter in the analyte; and an estimated percentage of the neurotransmitter relative to another neurotransmitter in the analyte.

In some embodiments of any of the above methods, the biosensor is selected from a plurality of different biosensors.

In some embodiments of any of the above methods, the plurality of different biosensors includes a first biosensor and a second biosensor that differ from one another in one or more of: plasmonic materials used in respective engineered layers; amplitudes of feature height variation in the respective engineered layers; transverse sizes of holes in the respective engineered layers; average distances between the holes; and geometric shapes of the holes.

In some embodiments of any of the above methods, the method further comprises loading the analyte onto the selected biosensor.

In some embodiments of any of the above methods, the loading comprises spin-coating the selected biosensor with a polyvinyl alcohol solution containing the analyte.

In some embodiments of any of the above methods, the method further comprises measuring the time series of fluorescence spectra of the loaded biosensor, with different ones of the fluorescence spectra in the time series corresponding to different respective illumination times.

A non-transitory computer-readable medium storing instructions that, when executed by a computing device, cause the computing device to perform operations comprising at least some of the above methods.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary.

While this disclosure includes references to illustrative embodiments, this specification is not intended to be construed in a limiting sense. Various modifications of the described embodiments, as well as other embodiments within the scope of the disclosure, which are apparent to persons skilled in the art to which the disclosure pertains are deemed to lie within the principle and scope of the disclosure, e.g., as expressed in the following claims.

Unless explicitly stated otherwise, each numerical value and range should be interpreted as being approximate as if the word “about” or “approximately” preceded the value or range.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

Unless otherwise specified herein, in addition to its plain meaning, the conjunction “if” may also or alternatively be construed to mean “when” or “upon” or “in response to determining” or “in response to detecting,” which construal may depend on the corresponding specific context. For example, the phrase “if it is determined” or “if [a stated condition] is detected” may be construed to mean “upon determining” or “in response to determining” or “upon detecting [the stated condition or event]” or “in response to detecting [the stated condition or event].”

Also, for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements.

The functions of the various elements shown in the figures, including any functional blocks labeled as “processors” and/or “controllers,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non volatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

As used in this application, the terms “circuit,” “circuitry” may refer to one or more or all of the following: (a) hardware-only circuit implementations (such as implementations in only analog and/or digital circuitry); (b) combinations of hardware circuits and software, such as (as applicable): (i) a combination of analog and/or digital hardware circuit(s) with software/firmware and (ii) any portions of hardware processor(s) with software (including digital signal processor(s)), software, and memory (ies) that work together to cause an apparatus, such as a mobile phone or server, to perform various functions); and (c) hardware circuit(s) and or processor(s), such as a microprocessor(s) or a portion of a microprocessor(s), that requires software (e.g., firmware) for operation, but the software may not be present when it is not needed for operation.” This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

It should be appreciated by those of ordinary skill in the art that any block diagrams herein represent conceptual views of illustrative circuitry embodying the principles of the disclosure. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

Any numerical range recited herein includes all values from the lower value to the upper value. For example, if a range is stated as 1% to 50%, it is intended that the narrower ranges thereof, such as 2% to 40%, 10% to 30%, 1% to 3%, etc., are expressly enumerated by said statement. These specific examples represent only a limited subset of what is intended to be covered, and all possible combinations of numerical values between and including the lowest value and the highest value of the enumerated range are to be considered to be expressly stated in this application. Concentration ranges, pH ranges, and other ranges of specific parameters are intended to be interpreted in a manner similar to the “%” example.

The modifier “about” or “approximately” used in connection with a quantity is inclusive of the stated value and has the meaning dictated by the context (for example, it includes at least the degree of error associated with the measurement of the particular quantity). The modifier “about” or “approximately” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so that, for example, “about 1” may also mean from 0.5 to 1.4.

For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, the present disclosure relies on general principles of organic chemistry, inorganic chemistry, and material science, as accepted in the pertinent arts. For example, specific functional moieties and reactivity in accordance with some of such principles are described in Organic Chemistry, Thomas Sorrell, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987, the entire contents of each of which are incorporated herein by reference.

“BRIEF SUMMARY OF SOME SPECIFIC EMBODIMENTS” in this specification is intended to introduce some example embodiments, with additional embodiments being described in “DETAILED DESCRIPTION” and/or in reference to one or more drawings. “BRIEF SUMMARY OF SOME SPECIFIC EMBODIMENTS” is not intended to identify essential elements or features of the claimed subject matter, nor is it intended to limit the scope of the claimed subject matter.

What is claimed is:

1. An analytical method, comprising:
determining a first rate constant based on a time series of fluorescence spectra of a biosensor having loaded thereon an analyte and subjected to illumination by ultraviolet light, the biosensor including an engineered layer of plasmonic material, the analyte including a neurotransmitter, the first rate constant corresponding to a photochemical reaction of the neurotransmitter caused by the ultraviolet light;
submitting to a database a query containing at least the determined first rate constant, the database including photobleaching calibration data representing a plurality of different neurotransmitters and a plurality of different biosensors; and
receiving from the database a response to the submitted query.

2. The method of claim 1, wherein the determining comprises:
fitting an exponential function to fluorescence decay kinetics represented by the time series; and
determining the first rate constant based on the fitting.

3. The method of claim 2, wherein the determining further comprises:
integrating over a range of wavelengths each of the fluorescence spectra; and
performing the fitting using the integrated fluorescence spectra.

4. The method of claim 2,
wherein the exponential function has first and second exponential components;
wherein the first rate constant corresponds to the first exponential component; and
wherein the determining further comprises determining, based on the fitting, a second rate constant corresponding to the second exponential component, a first amplitude corresponding to the first exponential component, and a second amplitude corresponding to the second exponential component.

5. The method of claim 4, wherein the query further contains at least one of the second rate constant, the first amplitude, and the second amplitude.

6. The method of claim 1, wherein the neurotransmitter is selected from the group consisting of:
a monoamine neurotransmitter (MANT);
dopamine (DA);
serotonin (SER);
norepinephrine (NE);
epinephrine (E);
histamine (H);
tryptophan (TRP); and
3,4-dihydroxyphenylacetic acid (DOPAC).

7. The method of claim 1, wherein the plasmonic material comprises a material selected from the group consisting of Al, Au, Ag, Cu, Ti, Cr, TiN, ZrN, HIN, VN, and NbN.

8. The method of claim 1, wherein the ultraviolet light causes both the fluorescence and the photochemical reaction.

9. The method of claim 1, wherein the engineered layer has a varying feature height across the biosensor.

10. The method of claim 1, wherein the response includes a characteristic selected from the group consisting of:
a predicted identity of the neurotransmitter;
a list of predicted identities of the neurotransmitter ranked based on respective confidence scores;
an estimated amount of the neurotransmitter in the analyte; and
an estimated percentage of the neurotransmitter relative to another neurotransmitter in the analyte.

11. The method of claim 1, wherein the biosensor is selected from a plurality of different biosensors.

12. The method of claim 11, wherein the plurality of different biosensors includes a first biosensor and a second biosensor that differ from one another in one or more of:
plasmonic materials used in respective engineered layers;
amplitudes of feature height variation in the respective engineered layers;
transverse sizes of holes in the respective engineered layers;
average distances between the holes; and
geometric shapes of the holes.

13. The method of claim 11, further comprising loading the analyte onto the selected biosensor.

14. The method of claim 13, wherein the loading comprises spin-coating the selected biosensor with a polyvinyl alcohol solution containing the analyte.

15. The method of claim 13, further comprising measuring the time series of fluorescence spectra of the loaded biosensor, with different ones of the fluorescence spectra in the time series corresponding to different respective illumination times.

16. A non-transitory computer-readable medium storing instructions that, when executed by a computing device, cause the computing device to perform operations comprising the method of claim 1.

17. An analytical apparatus, comprising:
a fluorimeter configured to measure a time series of fluorescence spectra of a biosensor having loaded thereon an analyte and subjected to illumination by ultraviolet light, the biosensor including an engineered layer of plasmonic material, the analyte including a neurotransmitter, different ones of the fluorescence spectra in the time series corresponding to different respective illumination times; and
a computing device configured to:
determine a first rate constant based on the time series, the first rate constant corresponding to a photochemical reaction of the neurotransmitter caused by the ultraviolet light;
submit to a database a query containing at least the determined first rate constant, the database including photobleaching calibration data representing a plurality of different neurotransmitters and a plurality of different biosensors; and
display on a display device a response to the submitted query received from the database.

18. The apparatus of claim 17, wherein the computing device is further configured to:
fit an exponential function to fluorescence decay kinetics represented by the time series; and
determine the first rate constant based on the fit.

19. The apparatus of claim 18, wherein the computing device is further configured to:
integrate over a range of wavelengths each of the fluorescence spectra in the time series; and
obtain the fit using the integrated fluorescence spectra.

20. The apparatus of claim 18,
wherein the exponential function has first and second exponential components;
wherein the first rate constant corresponds to the first exponential component;
wherein the computing device is further configured to determine, based on the fit, a second rate constant corresponding to the second exponential component, a first amplitude corresponding to the first exponential component, and a second amplitude corresponding to the second exponential component; and
wherein the query further contains at least one of the second rate constant, the first amplitude, and the second amplitude.
